(19) United States
(12) Patent Application Publication
(10) Pub. No.: US 2012/0129154 A1
(43) Pub. Date: May 24, 2012

(54) METHODS AND COMPOSITIONS FOR THE DETECTION OF BACTERIAL BLIGHT

(76) Inventors: David A. Schofield, Hollywood, SC (US); Carolee Bull, Salinas, CA (US); Ian J. Molineux, Austin, TX (US)

(21) Appl. No.: 12/953,612

(22) Filed: Nov. 24, 2010

(51) Int. Cl. C12Q 1/70; C12N 7/00

(52) U.S. Cl. 435/5; 435/235.1

(57) ABSTRACT

Methods and compositions relating to the detection of bacterial blight are provided. In one embodiment, a detection system is provided comprising a phage operable to infect a Pseudomonas cannabina and/or Pseudomonas syringae microorganism, the phage comprising a detectable reporter configured and arranged to be expressed upon infection of the microorganism by the phage, wherein the detectable reporter comprises nucleic acid encoding a luxAB gene; and a detector operable to detect expression of the luxAB reporter nucleic acid.

### STATEMENT OF GOVERNMENT INTEREST

The present invention was made with support under Grant Number 1012059 awarded by the National Science Foundation. The U.S. government has certain rights in the invention.

### BACKGROUND

Pseudomonas cannabina pv. alisalensis, previously known as Pseudomonas syringae pv. alisalensis, is the causative agent of bacterial blight, a disease afflicting cruciferous vegetables. Cruciferous vegetables are one of the most dominant food crops worldwide and are a valuable U.S. commodity; economically, crucifers have a U.S. value of over $1.3 billion annually. P. cannabina pv. alisalensis has been documented to cause severe bacterial blight in conventional and organic production fields in California, Nevada and New Jersey on members of the family Brassicaceae including: arugula, broccoli, cauliflower, rutabaga, brussel sprouts, and cabbage.

Initial symptoms of disease consist of small water-soaked flecks on the lower foliage. Over time, these flecks expand and become surrounded by bright yellow borders, which eventually coalesce to form large necrotic areas, rendering the crop unmarketable. The pathogen is transmissible and can be passed through soil inoculum, and there is evidence to suggest that P. cannabina pv. alisalensis may be seedborne. Therefore, it is essential to be able to rapidly and specifically identify the causative agent, P. cannabina pv. alisalensis before symptom onset otherwise severe crop damage can occur rendering the produce unmarketable.

Bacterial blight caused by P. cannabina pv. alisalensis has only recently been identified. Consequently, there are no commercially available or USDA-approved detection technologies. Standard bacterial detection methodologies which could be adapted include microbiological-, DNA-, and antibody-based techniques. However, microbiological based assays generally take 1-3 days to complete. In addition, DNA assays (e.g. PCR or DNA hybridizations) require expensive equipment and consumables, and those currently available are not sensitive enough for commercial use. Immunoanalysis offers the potential for rapid tests but tend to be problematic since the antibodies react with other closely related species and the monoclonal antibodies may not react with all strains. Moreover, both immunoanalysis and DNA methodologies detect the presence of antigen or DNA but provide no information on whether the cell is viable and potentially infectious.

### SUMMARY

The present disclosure, according to some embodiments, generally relates to compositions, methods, systems and kits for detection of microbes. In more specific embodiments, the present disclosure relates to compositions, methods, systems and kits for the detection and/or identification of biological pathogens of Pseudomonas species (e.g., Pseudomonas cannabina, Pseudomonas syringae, etc.) using phage binding and bacterial infection.

In one embodiment, the present disclosure provides a Pseudomonas cannabina and/or Pseudomonas syringae detection system comprising: a phage operable to infect a Pseudomonas cannabina and/or a Pseudomonas syringae microorganism, the phage comprising a detectable reporter configured and arranged to be expressed upon infection of the microorganism by the phage, wherein the detectable reporter comprises nucleic acid encoding a luxAB gene; and a detector operable to detect expression of the luxAB reporter nucleic acid.

In another embodiment, the present disclosure provides a phage operable to infect a Pseudomonas cannabina and/or Pseudomonas syringae microorganism comprising a detectable reporter configured and arranged to be expressed upon infection of the microorganism, the detectable reporter comprising a nucleic acid encoding a luxAB gene, and wherein the expression of the luxAB gene is detected as bioluminescent light.

In another embodiment, the present disclosure provides a method of detecting the presence of a Pseudomonas cannabina and/or Pseudomonas syringae microorganism in a sample comprising a) providing a phage operable to infect a Pseudomonas cannabina and/or Pseudomonas syringae microorganism, the phage comprising a detectable reporter configured and arranged to be expressed upon infection of the microorganism by the phage; b) contacting the sample with the phage under conditions that permits the phage to infect the microorganism and express the detectable reporter; and c) detecting expression of the detectable reporter, wherein detecting the detectable reporter indicates that the Pseudomonas cannabina and/or Pseudomonas syringae microorganism is present in the sample.

In yet another embodiment, the present disclosure provides a kit comprising: a) a phage operable to infect a Pseudomonas cannabina and/or Pseudomonas syringae microorganism, the phage comprising a detectable reporter configured and arranged to be expressed upon infection of the microorganism by the phage, in a suitable container; and b) one or more containers to mix the phage with a sample that may comprise the microorganism.

The features and advantages of the present invention will be apparent to those skilled in the art. While numerous changes may be made by those skilled in the art, such changes are within the spirit of the invention.

### DESCRIPTION

The present disclosure, according to some embodiments, generally relates to compositions, methods, systems and kits for detection of microbes. In more specific embodiments, the present disclosure relates to compositions, methods, systems and kits for the detection and/or identification of biological pathogens of Pseudomonas species (e.g., Pseudomonas cannabina, Pseudomonas syringae, etc.) using phage binding and bacterial infection.

In some embodiments, the present disclosure may provide the ability to detect and/or identify the presence of a Pseudomonas microorganism, and more specifically, Pseudomonas cannabina and/or Pseudomonas syringae. The present disclosure provides a significant advantage over other detection technologies for the routine screening of samples, such as seed populations, since PCR and immunoanalysis can produce false positives irrespective of viability leading to seed destruction and economic waste. According to certain embodiments, phage assays of the present disclosure may be rapid (e.g., minutes) and require minimal processing, sensitive (e.g., 10² CFU/mL), specific in that the assays may only detect viable (potentially infectious) cells, and require minimal consumables. In certain specific embodiments, the present disclosure provides methods capable of: (i) detecting P. cannabina pv. alisalensis from asymptomatic and diseased plant specimens, or from cultivated lab specimens, and (ii) detecting the pathogen from various inoculum sources such as seed, soil, water and weeds.

As mentioned above, the present disclosure relates, in some embodiments, to biological detection compositions, methods, systems and/or kits for rapid detection of a bacterial cell such as a P. cannabina and/or P. syringae cell. P. cannabina and P. syringae are rod shaped, Gram-negative bacteria with polar flagella. They are generally plant pathogens which can infect a wide range of plant species, and exists as over 50 different pathovars, including P. cannabina pv. alisalensis. The compositions, methods, systems and/or kits of the present disclosure, in some embodiments, may be used, for the detection of one or more Pseudomonas species exemplified in non-limiting examples by the plant pathogenic strains P. cannabina and P. syringae as well as mutations and genetically engineered variants thereof, etc.

In some embodiments, the compositions, methods, systems and/or kits of the disclosure, comprise a bacteriophage that is operable to infect a Pseudomonas microorganism. In some embodiments, one or more bacteriophage specific to P. cannabina and/or P. syringae may be used. For example, in one embodiment, a suitable phage may be PBS1. Other examples of suitable phages may include, but are not limited to, phages φ6, φ12, φ13, φ8, 9B, 123, 788/8, and φ2954.

In some embodiments, a bacteriophage operable to infect a Pseudomonas microorganism may comprise a detectable reporter. In some embodiments, a detectable reporter may comprise a nucleic acid which leads to the production of a detectable gene product. In some embodiments, a detectable gene product may comprise a protein that is encoded by the luxAB genes from Vibrio harveyi (GenBank Accession No. E12410, version 1, last updated Apr. 20, 2006). In some embodiments, a phage may be a genetically engineered phage comprising nucleic acids encoding a detectable gene product, e.g., a luxAB gene that encodes a luciferase enzyme.

In some embodiments, a detectable gene product may comprise or consist of a luciferase enzyme. Contacting a luciferase enzyme with a suitable luciferin substrate may produce bioluminescence. Substrates for a luciferase enzyme may comprise an aldehyde (e.g., n-decanal). In some embodiments, detection of bioluminescence upon binding or infection of the bacterial cell by the phage detects the presence of a Pseudomonas microorganism. The bioluminescent light signal may be visualized by a light detection device, or a simple hand-held photon-detection device; minimal processing of the sample may be required. In some embodiments, the light signal detected may be analyzed for different wavelengths. In some embodiments the detection of the detectable reporter gene product may be by PCR or immunological methods.

In some embodiments, the detection of bioluminescence may be achieved in a time of less than 90 minutes following infection with a recombinant phage of the disclosure. In some embodiments, the detection of bioluminescence, and hence of a Pseudomonas microbe, may be in a time less than 60 minutes, less than 40 minutes, less than 30 minutes, less than 25 minutes, less than 20 minutes, less than 18 minutes, less than 17 minutes, less than 16 minutes, less than 15 minutes, less than 14 minutes, less than 13 minutes, less than 12 minutes, less than 11 minutes, less than 10 minutes, less than 9 minutes, less than 8 minutes, less than 7 minutes, less than 6 minutes, less than 5 minutes, less than 4 minutes less than 3 minutes, less than 2 minutes to less than 1 minute, following infection with a recombinant phage of the disclosure.

The lux genes control bioluminescence in a wide variety of species including marine and terrestrial species such as bacteria, dinoflagellates, fungi, fish, insects, shrimp, and squid. Cloning and expressing lux genes from different species have led to significant advances in understanding the molecular biology of bioluminescence. Lux operons may have a common gene organization of luxCDAB(F)E, with luxAB coding for the enzyme luciferase and luxCDE coding for the fatty acid reductase complex responsible for synthesizing fatty aldehydes which are substrates for the luminescence reaction. However, significant differences exist in their sequences and properties as well as in the presence of other lux genes (such as lux I, R, F, G, and H). In some embodiments, a luciferase-encoding nucleic acid (e.g., luxAB from any species) may also be used in the compositions, methods, systems and/or kits of the disclosure. For example, a luxAB from Xenorhadbus luminescens may be used. Other non-limiting examples include a luxAB from V. fischeri, Photinus pyralis (firefly), Photobacterium sp., and Photorhabdus luminescens.

In some embodiments, a phage operable to infect a Pseudomonas microorganism, may comprise in addition to a reporter, nucleic acids encoding for the luxCDE genes (from any species), which encode a fatty acid reductase complex that may synthesize fatty aldehydes which are substrates for the luminescence reaction. This may eliminate the need to add aldehyde substrates to detect bioluminescence.

In some embodiments, expression of a recombinant phage of the present disclosure and light production may require a phage-infected bacterial cell being detected to have an active metabolism. In some embodiments, a phage reporter gene may be under the control of one or more transcriptional elements (such as but not limited to, promoters, enhancers, repressors, transcriptional terminators, etc.) and/or translational elements (such as but not limited to, ribosome binding sites). Posttranslational modifying elements may also be desirable. A reporter gene may comprise a nucleic acid encoding a detectable gene product as well as transcriptional and/or translational control elements for expression of the detectable gene product. Bacterial cellular machinery may be desirable for expression of a reporter gene comprising one or more bacterial transcriptional and/or translational elements. In some embodiments, production of the detectable gene product by the reporter nucleic acid may be dependent on one or more components of the bacterial cell that the phage is infecting. For example, expression of the reporter may indicate the presence of live and infectious bacteria as well as bacteria of a specific species. Since only viable cells produce a light signal, these example compositions, methods, systems and/or kits of the disclosure of the present disclosure detect viable and infectious bacterial cells. This is a distinct advantage over PCR detection methodologies which detect the presence of of a Pseudomonas microorganism, but yield no information as to whether the cells are viable and infectious.

In some embodiments, the present disclosure relates to a detection system comprising: (a) a phage operable to infect a Pseudomonas cannabina and/or Pseudomonas syringae microorganism, the phage comprising a detectable reporter configured and arranged to be expressed upon infection of the microorganism by the phage, and (b) a detector operable to detect reporter expression.

Expression of a detectable reporter, in some embodiments, may include transcription and/or translation. According to some embodiments, expression may include post transcriptional and/or posttranslational modification(s) of a gene product. In some embodiments, one may detect a detectable gene product that may be formed following phage binding and/or infection of a Pseudomonas cannabina and/or Pseudomonas syringae microorganism. For example, a detectable gene product may include a product of transcription (e.g., an RNA), a product of translation (e.g. a peptide or a protein), and/or a product of post transcriptional and/or posttranslational modification. A detectable gene product, in some embodiments, may include a product that may form as a result of phage binding (e.g., tagging a phage coat protein with a fluorescent moiety) or infection which does not require transcription and/or translation.

In some embodiments, the present disclosure also relates to methods for preparing a bacteriophage configured and arranged to detect a microbe. For example, a Pseudomonas luxAB reporter phage may be generated using the PBS1 phage. LuxAB may be cloned into an expression cassette under the transcriptional and translational control of preferred Pseudomonas expression sequences. The expression cassette may be flanked by PBS1 phage DNA to allow homologous recombination of the expression cassette into the phage DNA. LuxAB may be integrated into a non-coding region of the PBS1 genome by homologous recombination based on a double cross over event. Recombinant PBS1::luxAB may be identified and isolated based on the ability of infected cultures to emit light. LuxAB integration may be verified by diagnostic agarose gel electrophoresis and PCR. The ‘fitness’ of the recombinant phage may be compared to the wild-type phage.

The sensitivity of the bioluminescence assay may be from about 1 CFU/mL to 100,000,000 CFU/mL or about 1 CFU/mL to 10,000,000 CFU/mL or about 1 CFU/mL to 1,000,000 CFU/mL or about 1 CFU/mL to 100,000 CFU/mL or about 1 CFU/mL to about 50,000 CFU/mL or about 100 CFU/mL to 1000 CFU/mL or about 1 CFU/mL to about 100 CFU/mL. In some embodiments, the sensitivity may be about 1 CFU/mL, 2 CFU/mL, 3 CFU/mL, 4 CFU/mL, 5 CFU/mL, 6 CFU/mL, 7 CFU/mL, 8 CFU/mL, 9 CFU/mL to about 10 CFU/mL.

In some embodiments, the sensitivity may be about 1 CFU/mL, about 10 CFU/mL, about 20 CFU/mL, about 30 CFU/mL, about 40 CFU/mL, about 50 CFU/mL, 60 CFU/mL, about 70 CFU/mL, about 80 CFU/mL, about 90 CFU/mL to about 100 CFU/mL. In some embodiments, the sensitivity may be from about 100 CFU/mL to about 1000 CFU/mL and may be about 100 CFU/mL, about 200 CFU/mL, about 300 CFU/mL, about 400 CFU/mL, about 500 CFU/mL, about 600 CFU/mL, about 700 CFU/mL, about 800 CFU/mL, about 900 CFU/mL, to about 1000 CFU/mL. In some embodiments, the sensitivity may include values in between the ranges listed above.

In some embodiments, the sensitivity of the assay may be about 1000 CFU/mL to about 10,000,000 CFU/mL, and may be about 100,000 CFU/mL, about 200,000 CFU/mL, about 300,000 CFU/mL, about 400,000 CFU/mL, about 500,000 CFU/mL, about 600,000 CFU/mL, about 700,000 CFU/mL, about 800,000 CFU/mL, about 900,000 CFU/mL, about 1,000,000 CFU/mL, about 2,000,000 CFU/mL, about 3,000,000 CFU/mL, about 4,000,000 CFU/mL, about 5,000,000 CFU/mL, about 6,000,000 CFU/mL, about 7,000,000 CFU/mL, about 8,000,000 CFU/mL, about 9,000,000 CFU/mL, to about 10,000,000 CFU/mL.

In some embodiments, the sensitivity of the assay may be about 1000 CFU/mL to about 50,000 CFU/mL, and may be about 1000 CFU/mL, about 2000 CFU/mL, about 3000 CFU/mL, about 4000 CFU/mL, about 5000 CFU/mL, about 6000 CFU/mL, about 7000 CFU/mL, about 8000 CFU/mL, about 9000 CFU/mL, about 10,000 CFU/mL, about 15,000 CFU/mL, about 20,000 CFU/mL, about 25,000 CFU/mL, about 30,000 CFU/mL, about 35,000 CFU/mL, about 40,000 CFU/mL, about 45,000 CFU/mL to about 50,000 CFU/mL.

In some embodiments, a reporter may comprise a nucleic acid which leads to the production of a detectable gene product. In some embodiments, the reporter may comprise nucleic acids that lead to the production of a fluorescent protein such as a green fluorescent protein (GFP), which may be detected as a green fluorescent light when exposed to UV light. In some embodiments, the reporter may comprise nucleic acids that lead to the production of a GFP, a red fluorescent protein (DsRed), or a yellow fluorescent protein or mutations and variants thereof.

In some embodiments, the reporter may comprise nucleic acids that lead to the production of an ice nucleation gene (inaZ). In some embodiments, the reporter may comprise nucleic acids that lead to the production of the beta-glucuronidase (gusA), which may be detected by colorimetric enzyme assay of cell extracts or indicator plates.

In some embodiments, the reporter may comprise nucleic acids that encode a lacZ gene, which encodes an enzyme β-galactosidase. Cells expressing β-galactosidase turn blue color when grown on a medium that contains the β-galactosidase substrate (e.g., the analog X-gal) which may be detected colorimetrically.

In some embodiments, the reporter may comprise nucleic acids that encode selectable-marker reporter which may confer an antibiotic resistant phenotype on the bacteria expressing the marker gene, e.g., a reporter may encode a chloramphenicol acetyltransferase (CAT) gene which confers resistance to the antibiotic chloramphenicol.

In some embodiments, the present disclosure relates to compositions, methods, systems and/or kits for detecting the presence of Pseudomonas bacterial cells that may not (e.g. do not) require sample processing, extensive incubation periods, or a laboratory environment. Recombinant phage cells may be mixed with a test sample suspected of comprising a Pseudomonas microorganism and subsequently analyzed for bioluminescence. A suitable aldehyde substrate (e.g. n-decanal) may be also mixed in to obtain and/or enhance bioluminescence. A sample suspected of comprising a Pseudomonas microorganism may be any kind of a sample including seed, soil, water, weeds, etc.

Compositions, methods, systems and/or kits, according to some embodiments of the disclosure, may be configured to permit rapid detection of a microorganism, such as a Pseudomonas microorganism. For example, a Pseudomonas microorganism may be detected in less than about twelve (12) hours, less than about ten (10) hours, less than about eight (8) hours, less than about six (6) hours, or less than about four (4) hours. A target microorganism may be detected in less than about three (3) hours, less than about two (2) hours, or less than about one (1) hour. A target Pseudomonas microorganism may be detected in less than about forty minutes, less than about thirty minutes, less than about twenty minutes, less than about fifteen minutes, less than about thirteen minutes, less than about twelve minutes, less than about eleven minutes, less than about ten minutes, less than about nine minutes, less than about eight minutes, less than about seven minutes, less than about six minutes, less than about five minutes, less than about four minutes, less than about three minutes or less than about two minutes. As will be recognized by one of skill in the art, the time required for detection of a Pseudomonas microorganism may be a function of the time required for infection, and/or reporter expression and detection.

The present disclosure, in some embodiments, also relates to a kit for detecting Pseudomonas microorganisms. A kit, in some embodiments, may provide components necessary and/or desired for detecting a Pseudomonas microorganism in a sample. A diagnostic kit, according to some embodiments, may comprise a) a genetically engineered phage operable to infect a Pseudomonas microorganism, wherein the phage comprises a detectable reporter gene that is detectable only after phage infection of a Pseudomonas microorganism; b) a detector substrate that forms a detectable substrate upon expression of the reporter gene; and c) one or more containers to contact (e.g., mix), the phage with a sample that may comprise a Pseudomonas microorganism and detector substrate. Each component of the kit may be contained in a suitable container means such as a vial, tube, etc. and may be comprised in suitable solvents, buffers, or reagents. Alternatively some components may be present in a dry, powdered or lyophilized form. In some embodiments, a kit may also include suitable solvents, buffers and/or reagents required to reconstitute one or more component(s) as required.

In some embodiments, a kit of the present disclosure may comprise a) a genetically engineered PBS1 phage operable to infect a Pseudomonas microbe and comprising a luxAB reporter gene (PBS1::luxAB); b) a detector substrate for example, (e.g., an aldehyde such as n-decanal), that may react with the luxAB gene product to produce a detectable product (e.g. bioluminescent light); c) optionally a means for detecting the bioluminescent light. Each component may be packaged in suitable buffers, solutions or reagents and/or may be available as dry or lyophilized form.

In some embodiments, a kit according to the present disclosure may comprise a) a genetically engineered phage (e.g., PBS1) operable to infect P. cannabina and/or P. syringae, comprising a luxAB reporter gene (such as PBS1::luxAB) and a luxCDE gene; and b) a means for detecting bioluminescent light. Such a kit may optionally need small amounts of a detector substrate such as an aldehyde such as n-decanal, in case the luxCDE genes do not produce sufficient substrate that may react with the luxAB gene product to produce detectable bioluminescent light. Each component may be packaged in suitable buffers, solutions or reagents and/or may be available as dry or lyophilized form.

A kit, in some embodiments, may comprise one or more standard samples comprising Pseudomonas sp., for example, P. cannabina and/or P. syringae, for providing a measuring standard. Phage, (e.g., recombinant phage) may be resistant to environmental extremes and/or may be stored for months or years without a significant loss in phage infectivity. Bacterial cells however, may loose their viability and/or susceptibility to phage infection after storage for long periods of time. Thus, storage periods and storage conditions for components of a kit may vary.

A container may include any vessel into which a material may be placed (e.g., a vial, test tube, flask, bottles, syringe, pipette, and/or plate other container means). The individual containers of a kit may be maintained in close confinement (e.g., for commercial sale). Suitable larger containers may include injection or blow-molded plastic containers into which the desired vials are retained. Instructions and/or safety information may be provided with a kit.

Additionally, a bioluminescence detector such as a simple photodetector may be provided. A skilled artisan, having the benefit of the present disclosure, will recognize that any photodetector known in the art may be suitably used with the compositions, methods, detection systems and/or kits of the present disclosure.

As will be understood by those skilled in the art who have the benefit of the instant disclosure, other equivalent or alternative compositions, devices, methods, systems and/or kits for detecting Pseudomonas microorganisms or other bacterial microorganisms using bacteriophages can be envisioned without departing from the description contained herein. Accordingly, the manner of carrying out the disclosure as shown and described is to be construed as illustrative only. Persons skilled in the art may make various changes in the shape, size, number, and/or arrangement of parts without departing from the scope of the instant disclosure. For example, the location of a detectable reporter gene in the phage may be changed, and/or one or more different promoters and/or other expression/regulatory control sequences from those expressly described herein may be used. In some embodiments, a Pseudomonas expression/regulatory control sequence and/or a variant of a Pseudomonas expression/regulatory control element (such as promoter), and/or a expression/regulatory control element having a synthetic or semi-synthetic component may be used in accordance to the teachings herein. In another example, the type of a detectable reporter gene in the phage may be changed.

In addition, the size of a detection method, system and/or kit may be scaled up or down to suit the needs and/or desires of a practitioner. Also, where ranges have been provided, the disclosed endpoints may be treated as exact and/or approximations as desired or demanded by the particular embodiment. In addition, it may be desirable in some embodiments to mix and match range endpoints. A composition, method, system or kit may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A Pseudomonas cannabina and/or Pseudomonas syringae detection system comprising:
a phage operable to infect a Pseudomonas cannabina and/or Pseudomonas syringae microorganism, the phage comprising a detectable reporter configured and arranged to be expressed upon infection of the Pseudomonas cannabina and/or Pseudomonas syringae microorganism by the phage, wherein the detectable reporter comprises nucleic acid encoding a luxAB gene; and
a detector operable to detect expression of the luxAB reporter nucleic acid.

2. The detection system of claim 1, wherein the detectable reporter is operably linked to one or more Pseudomonas cannabina and/or Pseudomonas syringae expression control elements.

3. The detection system of claim 2, wherein the one or more expression control elements are selected from the group consisting of transcriptional control elements, translational control elements and combinations thereof.

4. The detection system of claim 1, wherein the microorganism is Pseudomonas cannabina pv. alisalensis.

5. The detection system of claim 1, wherein the phage is PBS1.

6. A phage operable to infect a Pseudomonas cannabina and/or Pseudomonas syringae microorganism comprising a detectable reporter configured and arranged to be expressed upon infection of the Pseudomonas cannabina and/or Pseudomonas syringae microorganism, the detectable reporter comprising a nucleic acid encoding a luxAB gene, and wherein the expression of the luxAB gene is detected as bioluminescent light.

7. The phage of claim 6, wherein the detectable reporter is operably linked to one or more Pseudomonas cannabina and/or Pseudomonas syringae expression control elements.

8. The phage of claim 6, wherein the microorganism is Pseudomonas cannabina pv. alisalensis.

9. The phage of claim 6, wherein the phage is PBS1.

10. A method of detecting the presence of a Pseudomonas cannabina and/or Pseudomonas syringae microorganism in a sample comprising:
a) providing a phage operable to infect a Pseudomonas cannabina and/or Pseudomonas syringae microorganism, the phage comprising a detectable reporter configured and arranged to be expressed upon infection of the Pseudomonas cannabina and/or Pseudomonas syringae microorganism by the phage;
b) contacting the sample with the phage under conditions that permits the phage to infect the Pseudomonas cannabina and/or Pseudomonas syringae microorganism and express the detectable reporter; and
c) detecting expression of the detectable reporter, wherein detecting the detectable reporter indicates that the Pseudomonas cannabina and/or Pseudomonas syringae microorganism is present in the sample.

11. The method of claim 10, wherein the phage is PBS1.

12. The method of claim 10, wherein the detectable reporter comprises nucleic acid.

13. The method of claim 12, wherein the nucleic acid encodes a luxAB gene.

14. The method of claim 10, wherein the detectable reporter is operably linked to one or more Pseudomonas cannabina and/or Pseudomonas syringae expression control elements.

15. The method of claim 10, wherein the one or more expression control elements are selected from the group consisting of transcriptional control elements, translational control elements and combinations thereof.

16. The method of claim 10, wherein detecting the expression of the detectable reporter comprises detecting bioluminescence.

17. The method of claim 16, wherein detecting bioluminescence further comprises providing a substrate specific to a luxAB gene product.

18. The method of claim 17, wherein the substrate comprises an aldehyde.

19. The method of claim 10, wherein the microorganism is Pseudomonas cannabina pv. alisalensis.

20. A kit comprising:
a) a phage operable to infect a Pseudomonas cannabina and/or Pseudomonas syringae microorganism, the phage comprising a detectable reporter configured and arranged to be expressed upon infection of the Pseudomonas cannabina and/or Pseudomonas syringae microorganism by the phage, in a suitable container; and
b) one or more containers to mix the phage with a sample that may comprise the Pseudomonas cannabina and/or Pseudomonas syringae microorganism.

21. The kit of claim 20, wherein the detectable reporter comprises nucleic acid encoding a luxAB gene.

22. The kit of claim 20, further comprising a detector substrate in a suitable container.

23. The kit of claim 20, further comprising a bioluminescence detector.

24. The kit of claim 20, wherein the phage is PBS1.

25. The kit of claim 20, further comprising a Pseudomonas cannabina and/or Pseudomonas syringae microorganism as a control.
